(19) United States
(12) Patent Application Publication
(10) Pub. No.: US 2026/0348939 A1
(43) Pub. Date: Nov. 5, 2026

(54) SYSTEMS AND METHODS FOR STORAGE OF DIGITAL INFORMATION VIA BIOPOLYMERS

(71) Applicants: Arizona Board of Regents On Behalf Of The University of Arizona, Tucson, AZ (US); Duke University, Durham, NC (US)

(72) Inventors: Frederic ZENHAUSERN, Phoenix, AZ (US); Devasier BENNET; Tuan VO-DINH, Durham, NC (US); Supriya ATTA, Durham, NC (US)

(21) Appl. No.: 19/099,330

(22) PCT Filed: Jul. 28, 2023
(86) PCT No.: PCT/US2023/028961

§ 371 Date: Jan. 28, 2025

Related U.S. Application Data
(60) Provisional application No. 63/393,476, filed on Jul. 29, 2022.
(60) Provisional application No. 63/394,901, filed on Aug. 3, 2022.

(51) Int. Cl. G01N 33/487; C12Q 1/6806; C12Q 1/6869

(52) U.S. Cl. CPC G01N 33/48721; C12Q 1/6806; C12Q 1/6869

(57) ABSTRACT

A method for storage of digital information via a biopolymer includes: receiving digital information; designing a target biopolymer sequence; encoding the digital information; synthesizing the target biopolymer sequence via a layered microfluidic device, the microfluidic device including: a pneumatic control layer configured to supply a control gas to a plurality of pneumatically operated valves; a fluidic layer comprising an interconnected matrix of microfluidic channels; and a biopolymer analysis layer comprising solid-state nanopores disposed in a semiconductor support. The method may also include: analyzing the target biopolymer sequence via the solid-state nanopores; transferring the target biopolymer sequence to a biopolymer preservation system; storing the target biopolymer sequence in the biopolymer preservation system; retrieving the target biopolymer sequence from the biopolymer preservation system; and decoding the target biopolymer sequence into the digital information.

### CROSS-REFERENCE TO RELATED APPLICATIONS

This application claims the benefit of priority to U.S. Provisional Patent Application No. 63/393,476, filed Jul. 29, 2022, and U.S. Provisional Patent Application No. 63/394,901, filed Aug. 3, 2022, each of which are hereby incorporated by reference in their entirety.

### BACKGROUND OF INVENTION

The amount of data generated globally has been increasing rapidly, and data storage capacity has not been keeping pace. Synthetic biopolymer (e.g., synthetic DNA) is a promising candidate as a digital storage medium, as it may achieve high density and successful long-term preservation. If non-biological information is to be stored and distributed over prolonged periods of time in the form of synthetic biopolymers such as DNA, artificial protection is required, because it degrades by various factors (for example, hydrolysis, water, UV irradiation, oxidation, ROS, ionizing radiation, heat, mutagenic chemicals nucleases, and pH). In most cases, DNA samples are stored at −80 °C or in liquid nitrogen (−196 °C), but there is a significant expense associated with maintaining these conditions as well as the associated contamination, degradation and fragmentation. Different storage media has been explored including DNA in solution, DNA in glass spheres, DNA in nanoparticles, and DNA in earth salt, but the stability, loading capacity, and handling are deficient in each of those systems (FIG. 1A).

Current biopolymer storage media is not optimal in all above aspects. There is an essential need of next generation storage medium by overcoming all the existing challenges, especially for end-to-end programmable automated biopolymer storage and preservation of biopolymer data. Previous biopolymer data storage studies in solution have shown that degradation rates can be accurately predicted, and buffer conditions can be adjusted to obtain stability at room temperature. However, those approaches are not reliable as they cannot handle errors efficiently and do not overcome the practical problems associated with physically storing the biopolymers to maintain stability over time.

Thus, it can be seen from the foregoing that improved systems and methods for biopolymer-based digital information storage are needed.

### SUMMARY OF THE INVENTION

Provided herein are systems and methods for storage of digital information via synthetic biopolymers. In some embodiments, the disclosed systems and methods may be characterized by complete automation in the write to store to read cycle of data storage, high biopolymer data loading, increased biopolymer stability, and simple sample handling (e.g., simple physical storage and accessibility).

In one embodiment, a system for storage of digitized information via a biopolymer may comprise a layered microfluidic device and a processor operably connected to the microfluidic device. The layered microfluidic device may comprise a pneumatic control layer, a fluidic layer, and a biopolymer analysis layer. The pneumatic control layer may be configured to supply a control gas to a plurality of pneumatically operated valves. The fluidic layer may comprise an interconnected matrix of microfluidic channels, the microfluidic channels being in selective fluid communication with each other via the plurality of pneumatically operated valves, a first side of the fluidic layer being bonded to the pneumatic control layer. The biopolymer analysis layer may comprise solid-state nanopores disposed in a semiconductor support, the solid state nanopores being in selective fluid communication with the interconnected matrix of microfluidic channels, the biopolymer analysis layer being bonded to a second side of the fluidic layer opposite the first side. The processor may be configured to cause the device to: receive digital information; design one or more target biopolymer sequences encoding the digital information; synthesize the one or more target biopolymer sequences via a reaction sequence carried out in the interconnected matrix of microfluidic channels and controlled via the plurality of pneumatically operated valves; analyze the one or more target biopolymer sequences via the solid-state nanopores; transfer the one or more target biopolymer sequences to a biopolymer preservation system; store the biopolymer; retrieve the one or more target biopolymer sequences from the biopolymer preservation system; and decode the one or more target biopolymer sequences into the digital information.

In one embodiment, the biopolymer is a nucleic acid and the target biopolymer sequence is a nucleotide sequence. In one embodiment, the semiconductor support is silicon. In one embodiment, the supercritical fluid is supercritical nitrogen or supercritical argon.

In one embodiment, the biopolymer preservation system comprises a dehydration channel, the dehydration channel being operably connected to a mineralization medium source and a supercritical fluid source, the processor being configured to cause the one or more target biopolymer sequences to be contacted with the mineralization medium and the supercritical fluid, thereby calcifying and dehydrating the one or more target biopolymer sequences.

In one embodiment, the mineralization medium comprises one or more of: Ca3(PO4)2, CaCl2·2H2O, and K2HPO4. In one embodiment, the mineralization medium comprises osteopontin and/or NaOH. In one embodiment, at least some of the microfluidic channels include one or more magnetic microspheres trapped therein via an externally applied magnetic field, and wherein a surface of each microsphere is functionalized to grow a target biopolymer sequence thereon.

In one embodiment, the digital information comprises binary computer code. In one embodiment, the nucleic acid comprises deoxyribonucleic acid (DNA) or ribonucleic acid (RNA). In one embodiment, the nucleic acid is selected from the group consisting of: α-I-threofuranosyl nucleic acid (TNA), 1′,5′-anhydrohexitol nucleic acid (HNA), arabino nucleic acid (ANA), 2′-fluoroarabino nucleic acid (FANA), cyclohexenyl nucleic acids (CeNA), and α-L-threofuranosyl nucleic acid.

In one embodiment, each solid-state nanopore has an effective diameter that is less than 50 nm.

In one embodiment, a method for storage of digital information via a biopolymer comprises: receiving digital information; designing one or more target biopolymer sequences, the one or more target biopolymer sequences encoding the digital information; synthesizing the one or more target biopolymer sequences via a reaction sequence carried out in an interconnected matrix of microfluidic channels of a layered microfluidic device. The device may comprise: a pneumatic control layer configured to supply a control gas to a plurality of pneumatically operated valves; a fluidic layer comprising an interconnected matrix of microfluidic channels, the microfluidic channels being in selective fluid communication with each other via the plurality of pneumatically operated valves, a first side of the fluidic layer being bonded to the pneumatic control layer; and a biopolymer analysis layer comprising solid-state nanopores disposed in a semiconductor support, the solid state nanopores being in selective fluid communication with the interconnected matrix of microfluidic channels, the biopolymer analysis layer being bonded to a second side of the fluidic layer opposite the first side and controlled via the plurality of pneumatically operated valves. The method may further comprise: analyzing the one or more target biopolymer sequences via the solid-state nanopores; transferring the one or more target biopolymer sequences to a biopolymer preservation system; storing the one or more target biopolymer sequences in the biopolymer preservation system; retrieving the one or more target biopolymer sequences from the biopolymer preservation system; and decoding the one or more target biopolymer sequences into the digital information.

In one embodiment, the biopolymer is a nucleic acid and the target biopolymer sequence is a nucleotide sequence. In one embodiment, the semiconductor support is silicon.

In one embodiment, storing the one or more target biopolymer sequences comprises contacting the one or more target biopolymer sequences with a mineralization medium. In one embodiment, the mineralization medium comprises one or more of: Ca3(PO4)2, CaCl2-2H2O, and K2HPO4. In one embodiment, the mineralization medium comprises osteopontin and/or NaOH. In one embodiment, storing the one or more target biopolymer sequences comprises contacting the one or more target biopolymer sequences in the mineralization medium with a supercritical fluid, thereby forming dehydrated target biopolymer sequences adsorbed on a mineral matrix. In one embodiment, the supercritical fluid is supercritical nitrogen or supercritical argon.

In one embodiment, at least some of the microfluidic channels include one or more magnetic microspheres trapped therein via an externally applied magnetic field, and wherein a surface of each microsphere is functionalized to grow a target biopolymer sequence thereon.

In one embodiment, the digital information comprises binary computer code. In one embodiment, the biopolymer is a nucleic acid. In one embodiment, the nucleic acid comprises deoxyribonucleic acid (DNA) or or ribonucleic acid (RNA). In one embodiment, the nucleic acid is selected from the group consisting of: α-I-threofuranosyl nucleic acid (TNA), 1′,5′-anhydrohexitol nucleic acid (HNA), arabino nucleic acid (ANA), 2′-fluoroarabino nucleic acid (FANA), cyclohexenyl nucleic acids (CeNA), and α-L-threofuranosyl nucleic acid.

In one embodiment, each solid-state nanopore has an effective diameter that is less than 50 nm. In one embodiment, the mineralization medium comprises a UV protection agent. In one embodiment, the UV protection agent is TiO2. In one embodiment, the mineralization medium comprises an ionizing radiation protection agent. In one embodiment, the ionizing radiation protection agent includes nanoparticles comprising Au, Sn, Sb, W, and/or Bi.

In one embodiment, storing the one or more target biopolymer sequences comprises incorporating the one or more target biopolymer sequences into a nanoparticle system, the nanoparticle system comprising nanoparticles of a metal-organic framework. In one embodiment, storing the one or more target biopolymer sequences comprises incorporating the one or more target biopolymer sequences into a nanoparticle system, the nanoparticle system comprising nanoparticles of a metal-organic framework coated with CeO₂. In one embodiment, the nanoparticles comprise a gold layer at least partially encapsulating the nanoparticle.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1A: DNA-based information storage system.

FIG. 1B: DNA-based information storage in biomimetic bone (synthetic fossils).

FIG. 2: Photograph of a synthetic bone matrix dried using supercritical nitrogen (left) and air dried (right).

FIG. 3A: Schematic of the microfluidic automaton integrated with the Solid-State Nanopores in Microfluidic chip.

FIG. 3B: Schematic diagram of a layered microfluidic device with microvalve network.

FIG. 3C: Cross-sectional view of the well interconnection showing lifting gate microfluidic features.

FIG. 4A: Overview of NA based data storage. Constitutional structures for the linearized backbone of DNA, TNA, HNA, FANA and CeNa (from left to right, respectively)

FIG. 4B: Synthesis cycle for on-chip implementation.

FIG. 5: Schematic diagram of one embodiment of the layered microfluidic device (left) and one embodiment of a continuous growth sequence for production of a biopolymer wherein steps 7-9 are repeated as necessary.

FIG. 6: Schematic illustration of the biopolymer preservation process.

FIG. 7: Biopolymer-based information storage in biomimetic bone (synthetic fossils)

FIG. 8: Example of radiation protecting DNA storage media

FIG. 9: Schematic illustration of protection of DNA from any external forces like pressure, introducing organic solvents, heating/cooling and from radiation, when the DNA is incorporated into a nanoparticle assembly including gold nanoparticles decorated onto CeO₂ coated MOF (ZIF-90).

FIG. 10: Schematic diagram of a method for analyzing biopolymer storage methods.

FIG. 11A: Schematic diagram of one embodiment of gDNA extraction and purification.

FIG. 11B: Schematic diagram of one embodiment of synthetic DNA synthesis.

FIG. 12: Schematic diagram of one embodiment of DNA encapsulation using Xanthan Gum Framework-Encoded Mineralization of Calcium Phosphate.

FIG. 13: Schematic diagram of one embodiment of DNA adsorption and desorption cycles on a synthetic bone matrix.

FIG. 14: Series of images showing environmental effects on a DNA encapsulated matrix.

FIG. 15: Characterization of a synthetic DNA encapsulation.

FIG. 16: Characterization of a gDNA adsorption/desorption cycle.

FIG. 17: Series of images showing Adsorption and Desorption efficiency.

FIG. 18A: XRD patterns of crystallization of DNA in the presence of CaP-XG.

FIG. 18B: FTIR Spectra of fingerprint analysis of immobilized DNA.

FIG. 19: Images showing DNA fragmentation/denaturation.

FIG. 20: Data showing analysis of fragmentation/denaturation and oxidative damage.

FIG. 21: Background subtracted Raman spectra of native DNA. The Raman modes of the DNA marked in this figure are mentioned in Table 1.

FIG. 22A: Representative Raman spectra of the DNA sample after ca-salt encapsulation.

FIG. 22B: Ten times zoomed in Raman spectra.

FIG. 23A: Representative Raman spectra of the 1st cycle, 2nd cycle, and 3rd cycle.

FIG. 23B: Raman spectra of these DNA samples where the Raman spectra were ten-fold zoomed in.

FIG. 24A: Representative Raman spectra of the DNA sample after temperature exposure.

FIG. 24B: Ten-fold zoomed in Raman spectra.

FIG. 25A: Representative Raman spectra of the DNA sample after UV-exposure.

FIG. 25B: Ten times zoomed in Raman spectra.

### STATEMENTS REGARDING CHEMICAL COMPOUNDS AND NOMENCLATURE

In general, the terms and phrases used herein have their art-recognized meaning, which can be found by reference to standard texts, journal references and contexts known to those skilled in the art. The following definitions are provided to clarify their specific use in the context of the invention.

As used herein, the term “digital information” refers to data that is stored, transferred, read, and used by networks, computers, and other machines. In some embodiments, digital information may be in the form of binary computer code.

As used herein, the term “operably connected” refers to a configuration of elements, wherein an action or reaction of one element affects another element, but in a manner that preserves each element's functionality. The connection may be by a direct physical contact between elements. The connection may be indirect, with another element that indirectly connects the operably connected elements. The term also refers to two or more functionally-related components being coupled to one another for purposes of flow of electric current and/or flow of data signals. This coupling of the two or more components may be a wired connection and/or a wireless connection. The two or more components that are so coupled via the wired and/or wireless connection may be proximate one another (e.g., in the same room or in the same housing) or they may be separated by some distance in physical space (e.g., in a different building).

As used herein, the term “target biopolymer sequence” refers to a sequence of biopolymer units that encodes a particular set of digital information.

As used herein, the term “biopolymer preservation system” refers to a system configured to prepare an amount of biopolymer for long-term storage. In some embodiments, the biopolymer preservation system may include a dehydration channel, wherein the dehydration channel is operably connected to a mineralization medium source and a supercritical fluid source. For example the biopolymer preservation system may comprise a microfluidic device operably connected to a reservoir of the mineralization medium and/or a reservoir of the supercritical fluid. In some embodiments, the biopolymer preservation system may be operably connected to a processor, wherein the processor is configured to control the biopolymer preservation system, including controlling the flow of the supercritical fluid as well as the mineralization medium. In some embodiments, the biopolymer preservation system may comprise PDMS. In some embodiments, the biopolymer preservation system may be an integral component of the layered microfluidic device configured to synthesize the biopolymer sequences.

As used herein, the term “effective diameter” refers to a characteristic dimension of an orifice or opening. The effective diameter, D, may be determined from the equation A=pi/4*D², where A is the cross sectional area of the opening. This reflects that the disclosed system and methods can accommodate nanopores that may not be exactly circular in cross-section.

In an embodiment, a composition or compound of the invention, such as an alloy or precursor to an alloy, is isolated or substantially purified. In an embodiment, an isolated or purified compound is at least partially isolated or substantially purified as would be understood in the art. In an embodiment, a substantially purified composition, compound or formulation of the invention has a chemical purity of 95%, optionally for some applications 99%, optionally for some applications 99.9%, optionally for some applications 99.99%, and optionally for some applications 99.999% pure.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

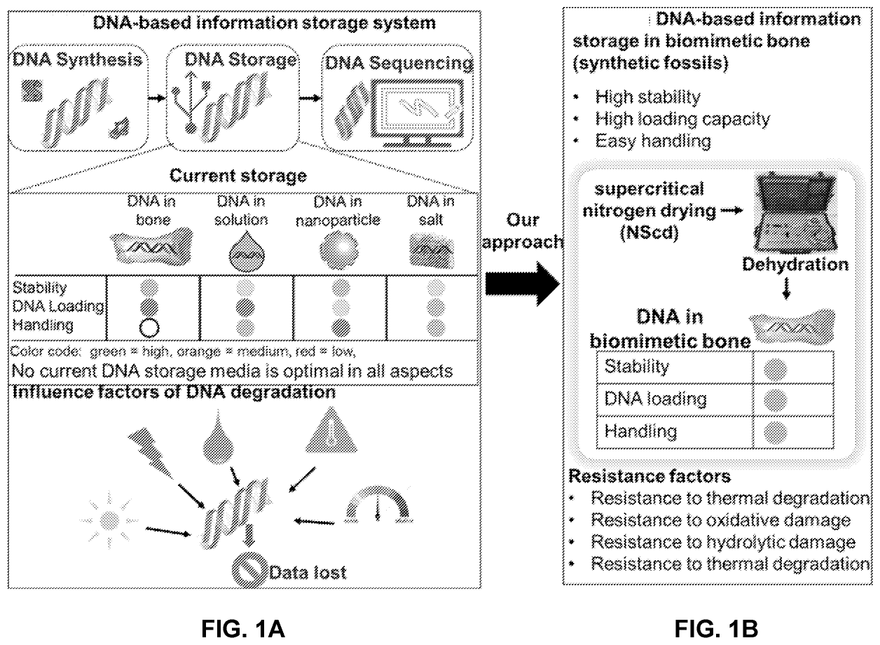

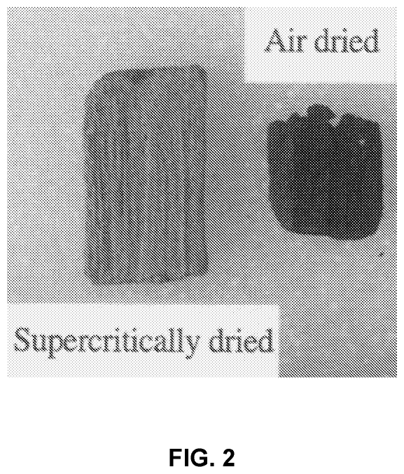

The disclosed systems may utilize programmable microfluidic platform (PMPs), to resolve all existing drawbacks for write to store to read cycle of data storage. In one aspect, biopolymers may be designed and synthesized to store digital information, then the biopolymers may be stored in a synthetic bone matrix after being mineralized and dehydrated via supercritical fluids such as nitrogen or argon (FIG. 1B) for solid-state preservation. It has been found that removing liquid in a precise and controlled way has a significant effect on the long term stability of the biopolymers in storage. Supercritical fluid dehydration can be rapidly accomplished at a low temperature, limiting crystal growth and producing three-dimensional (3D) organized structures (origami) with uniform size distributions. Samples dried via supercritical nitrogen drying (ScND), retained their original dimensions, whereas air-dried sample exhibited total structural collapse (FIG. 2). Thus, the supercritical drying technique may eliminate distortion to ultra-structure caused by drying effects. Biopolymer preservation in the solid-state bone matrix may facilitate room temperature storage, thereby significantly reducing cost and increasing convenience. In this matrix, high-capacity biopolymers can be kept stable enough to be sequenced after thousands of years of storage.

In one aspect, the multilayer PMP (PDMS-based microfluidic device) with automated Solid-State Nanopores in Microfluidic chip may operate the write to store to read cycle of data and analysis automatically.

Room temperature preservation of digital information on DNA in mineralized bone matrix is investigated. The synthetic bone is primarily composed of organic (collagen) and inorganic component (carbonated hydroxyapatite (HA), made up of various salts); the DNA may be protected in biomimetic bone (synthetic fossils) by biomimetic bone matrix mineralization and dehydration using ScND. The biomimetic mineralization has been frequently applied to design materials for bone regeneration, but instead of cell components, the DNA within the mineralized matrix is employed for DNA protection.

DNA molecules suffer from many problems such as inferior biophysical stability and propensity toward aggregation or degradation in the presence of external forces like heating, cooling, and especially from electromagnetic radiation. Accordingly, the disclosed systems and methods provide a robust and protective media for long-term reservation and protection of DNA data from exposure to ultraviolet light to ionizing radiation.

DNA is a relatively fragile biomolecule without any protection, prone to destabilization by environmental factors (for example, by hydrolysis or temperature, water, UV irradiation, oxidation, and pH).¹ The prevention of DNA degradation is possible, for instance, by storing DNA in a dried and anaerobic environment or at very low temperatures. However, there is a significant expense associated with maintaining these conditions and contamination/degradation/fragmentation that represents the primary threat to DNA preservation, and longevity and half-life of digital information are scarce. Storing DNA in a dehydrated (dried-state) with high loading capacity is the challenge. To overcome these existing issues (stability, loading capacity, and handling simplicity), DNA is dried using Supercritical nitrogen drying (NScD). Supercritical fluid drying (NScD) is an attractive alternative dehydration method because dehydration can be rapidly accomplished at a low temperature, limiting crystal growth and producing small particles with uniform size distributions. When the sample is dried under NScD retained its original dimensions, whereas if it is air-dried, resulting in total structural collapse.

On the other hand, using the prior art technology, the write to store to read cycle of DNA data is time-consuming, extremely expensive, and requirement of sophisticated instruments. The key challenges are complex procedures, risky solvent involvement, bio-hazardous chemical, difficulty in removing the solid support from the microfluidic device, and the requirement of sophisticated analytical instruments. The disclosed systems and methods provide a programmable microfluidic platform (PMP), which is automated, programmable, versatile sample processing and analysis various biomolecules can be ease. The PMP enables significant advances in the utility of microfluidics for chemical, biochemical, and biomolecule analysis, and synthesis including genetic analysis. The PMPs typically involves a complex sequence of steps to perform metering, mixing, thermal cycling, transferring and analysis of samples. Microvalves, and their use in arrays to fashion microfluidic pumps enables the fluidic control required to realize a programmable automated platform. Thus, in some embodiments PMPs are utilized to resolve all existing drawbacks for write to store to read cycle of DNA data. Thus simplifying the complexity of the synthesis cycle and subsequent analysis through the fully automated microfluidic-based device.

In one aspect, the disclosed device may reduce the total coupling and cleavage time for biopolymer synthesis. The device may be chemically resistant, capable of continuous-flow production, and suitable for the multiplex reactions. Furthermore, a solid support for facilitating the growth of biopolymer chains may be included in the micro device. For example, magnetic microspheres may be disposed in specific microvalves and trapped via an external magnetic field without to reduce loss of the biopolymer and disruptions to production.

In one aspect, in addition to its potential fluid manipulation, additional benefits of the process are the complete coupling of each amino acid and the complete removal of the excess of reagent from the growing DNA on the solid support.

In one embodiment, the solid phase oligonucleotide synthetic method may be utilized in the disclosed systems and methods. This approach remains unchanged from the introduction. The synthesis cycle involves deprotection, washing, coupling, and cleavage with a strong acid, which is highly risky and corrosive in nature. Accordingly, the disclosed systems and methods may utilize an acid-free cleavage cocktail.⁶ In some embodiments, superparamagnetic core-shell particles may be used as a solid phase support for biopolymer synthesis in the micro device. The superparamagnetic particles may be trapped in the reaction area by applying a magnetic field. After the complete cycle, the sample may be transferred into the automated capillary zone layer for nanopore analysis. The PMP microreactor can enable the multiplex reactions, thus is may be suitable for synthesis of biopolymers, implementing an error-correcting strategy, and combinatorial chemistry (write to store to read cycle of data).

In some embodiments, the solid-state nanopores of the analysis layer of the layered microfluidic device may have effective diameters less than 20 nm. In some embodiments, the solid-state nanopores of the analysis layer of the layered microfluidic device may have effective diameters less than 50 nm. The solid-state nanopores may be disposed in a silicon support to create a microfluidic network capable of sensing single DNA molecules at high bandwidths and with low noise.

_Ultraviolet (UV) light is well known to damage DNA by initiating a reaction between two molecules of thymine, one of the bases that make up DNA. Ultraviolet radiation (UVR) (mainly UV-B: 280-315 nm) is one of the powerful agents that can alter the normal state of life by inducing a variety of mutagenic and cytotoxic DNA lesions such as cyclobutane-pyrimidine dimers (CPDs), 6-4 photoproducts (6-4 PPs), and their Dewar valence isomers as well as DNA strand breaks by interfering the genome integrity. [Ref: [1A, 2A]. Alpha particles, beta particles and X-rays can directly affect a DNA molecule in one of three ways: 1) changing the chemical structure of the bases; 2) breaking the sugar-phosphate backbone; or 3) breaking the hydrogen bonds connecting the base pairs. Accordingly, the disclosed systems and methods may address this issue via one or more UV protection agents.

In one aspect, the microfluidic platform may be particularly well-suited for microscale chemical synthesis, as it permits discretized sample handling, allowing for total process control. Accordingly, in some embodiments, the microfluidic device may integrate PMP with in-line analysis via automated solid-state nanopores in the microfluidic chip.

The biopolymer read in automated solid-state nanopores in microfluidic chip platform that utilize nano scale structure, the chip dimensions can be selected to fit those of the targeted sample outlet and analysis object. This systematic approach will be able to read the compounds. Accordingly, the disclosed systems and methods may dramatically increase the sensitivity and accuracy while reducing the time, sample preparation volume, and cost. Further benefits include integrative sample analysis and the ability to rapidly and precisely read through the specific dielectric membranes with high sensitivity.

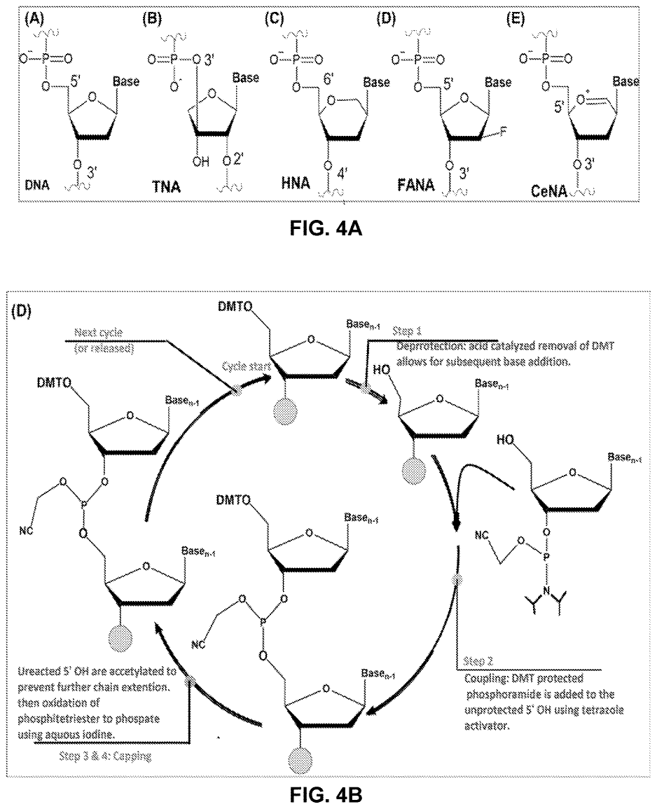

In some embodiments, synthetic biopolymers that are chemically more stable than DNA may be employed. For example, α-I-threofuranosyl nucleic acid (TNA),⁸ a nuclease-resistant nucleic acid, offers a biologically durable alternative for data archiving. Likewise (FIG. 4A), 1′,5′-anhydrohexitol nucleic acid (HNA),⁹ arabino nucleic acid (ANA),¹⁰ 2′-fluoroarabino nucleic acid (FANA),¹¹ cyclohexenyl nucleic acids (CeNA),⁹ and α-L-threofuranosyl nucleic acid,¹² may be employed for data archiving. A standard oligonucleotide synthesis cycle may be used for on-chip oligonucleotide synthesis with modified reagents. The solid phase oligonucleotide synthetic method is utilized (FIG. 4B). The synthesis cyclic involves deprotection, washing, coupling, and cleavage. The superparamagnetic core-shell particle may be used as a solid phase support, which can be retained in the reaction area by applying a magnetic field. Furthermore, magnetic separation may achieve high purity biopolymers. After the complete cycle, the sample goes into the automated solid-state nanopores in a silicon support into a microfluidic network, which can sense single DNA molecules.

### Example 1—Programmable Microfluidic Reactor

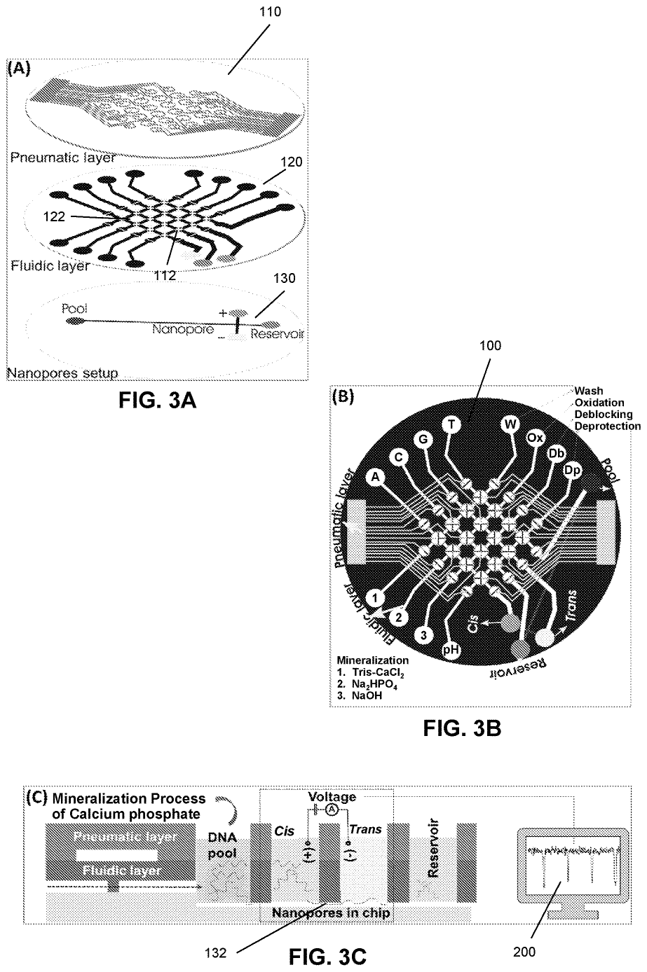

Chip fabrication consists of three steps. The modified chip design is patterned onto the wafer using lithography and wet etching. Then the patterned including the fluidic layer and the pneumatic control layer is molded by imprinting. The two-layer PDMS-based microfluidic chip is bonded, and the bottom of the two-layer chip is bonded with solid-state nanopores with effective diameters <20 nm in a silicon support into a microfluidic chip, which is used for sample analysis. FIG. 3 shows the design for the microfluidic reactor platform, is used for the fluidic layer (FIG. 3A—middle), and pneumatic layer (FIG. 3A—top) and solid-state nanopores setup (FIG. 3A—bottom). As can seen in the embodiment of FIG. 3, a system for storage of digital information via biopolymers may comprise a layered microfluidic device 100 and a processor 200. The layered microfluidic device 100 may comprise: a pneumatic control layer 110 configured to supply a control gas to a plurality of pneumatically operated valves 112; a fluidic layer 120 comprising an interconnected matrix of microfluidic channels 122 and a biopolymer analysis layer 130 comprising solid-state nanopores 132 disposed in a semiconductor support. The layered microfluidic device 100 may be operably connected to the processor 200. National Instruments LabVIEW control software is used for controlling the oligonucleotide synthesis. The software is configured to control the device, including, cyclic movement of fluids via the fluidic layer by controlling the pneumatic values.

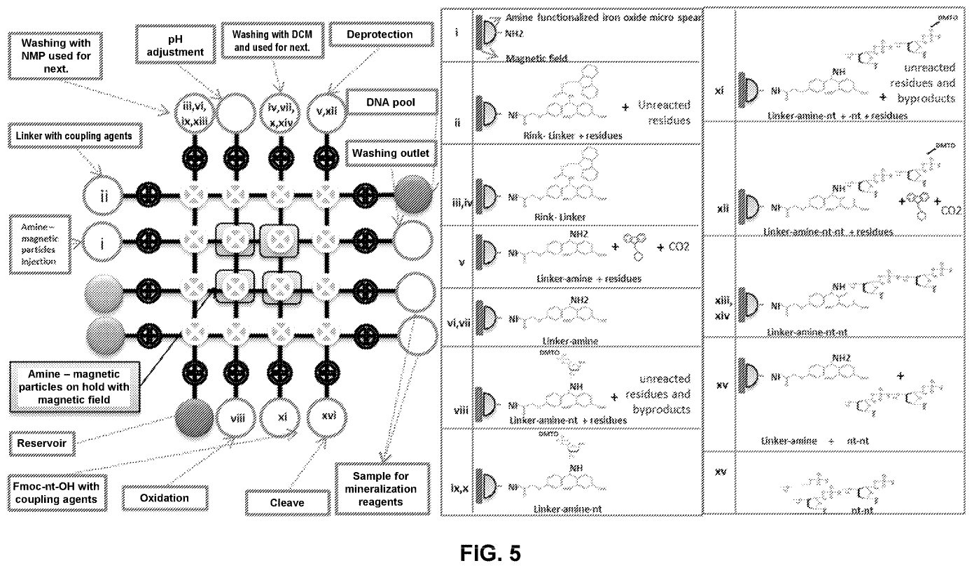

The time for one cycle and number of cycles is a function of the desired end products. Ongoing synthesis and end products are analyzed in the chip using solid-state nanopores. The on-chip model oligonucleotide sequence synthetic schemes are depicted in FIG. 5. On-chip standardization will be demonstrated with the A, C, G and T sequence, and then followed by huge numbers of new, synthetic and series of predefined and short sequences will be synthesized including synthesis and implement an error-correcting strategy. All the end products will be analyzed in the chip using solid-state nanopores and compare with standard nanopores (Minion) device. These oligonucleotide sequence will be used to apply for the development of a solid-state preservation of DNA data using SCF drying.

### Example 2 Solid-State Preservation of Digitally Encoded Biopolymers in Synthetic Bone Fossils Using SCF Drying

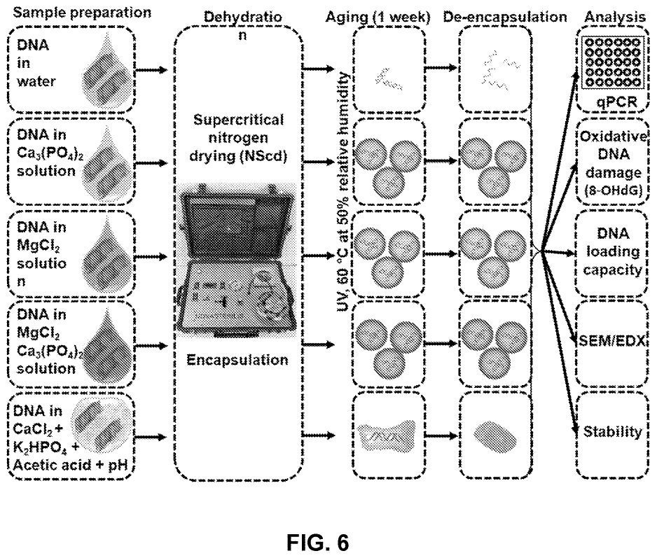

FIG. 6 depicts a method for encapsulation of DNA into various matrices via supercritical nitrogen drying/dehydration (ScND). After the de-encapsulation of DNA from matrices, the DNA is analyzed for stability, loading capacity, and handling efficiency.

Preparation of solutions: Ca₃(PO₄)₂, CaCl₂)·2H₂O and K₂HPO₄ each. 1M solution in water. Use 1:1 ratio for multiple solution and modified from.¹³

DNA Preparation: Prior to using, DNA is desalted and diluted to a final concentration of 15 ng/μl with water.

Sample preparation: Load 2 μl of DNA solution (15 ng/μl) in an Eppendorf tube, then add 5 μl of Ca₃(PO₄)₂, CaCl₂)·2H₂O and K₂HPO₄ solution respective tubes, individually enveloped in a pouch and heat sealed. All sealed pouches designed to be positioned inside the vessel of a NovaGenesis500 (NovaSterilis) instrument. Then the instrument is sealed with 25-foot pounds of torque. Then the solutions are dehydrated in a ScND for at least 2 hours. Vessel pressure is 1500 psi at 30 °C for periods of time.

De-encapsulation: To retrieve the DNA from the Eppendorf tube, 100 μl of a 1 mM EDTA solution is added to the tube and vortexed. For qPCR the sample is diluted additionally 1:100 to prevent interference of the salts in the amplification.

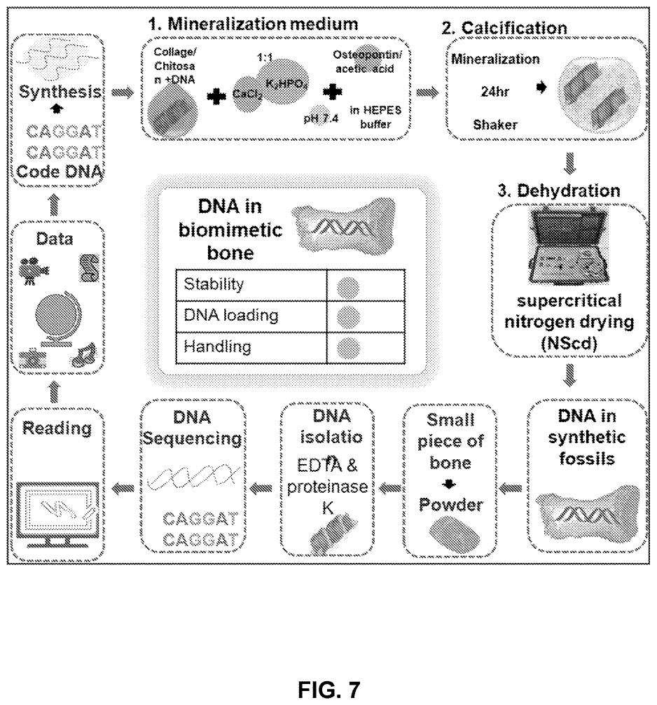

Bone matrix mineralization: (FIG. 7-1)

In order to induce mineralization of collagen/chitosan in the presence of DNA, a modified mineralization medium is prepared by mixing equal volumes of CaCl₂)·2H₂O and K2HPO4 solution in HEPES. Add osteopontin (100 μg/mL)/acetic acid (0.6%) to serve as the mineralization-directing agent in the CaCl₂) containing solution before the addition of K2HPO4. Ensure stable pH at 7.4 by adding 5 M NaOH to the solution. Incubate the samples under continuous agitation in a shaker for 24 hr to ensure uniform mineralization and complete calcification throughout the matrix.

For DNA damage analyse: 8-oxo-dG and anti-8-nitroguanine antibody are used for Dot immunobinding assay, which can potentially see the mutagenic DNA lesion, leading to the transversion of G:C to T:A, oxidative DNA damage and nitrative DNA damage.

Extract the total DNA and add 2 μg of DNA for the DNA oxidation blot. Briefly, dissolve DNA in nuclease free water and denatured at 65 °C for 5 min. Spot the samples on activated PVDF membrane, dry at room temperature (dark), and cross-linked under UV light for 30 min. Block the membranes in 5% milk (in 1×TBST) at room temperature for 1 h and incubate with 8-oxo-dG primary antibody (1:1,000) dilution at 4 °C overnight. On the next day, incubate the membrane in 1:10,000 anti-mouse secondary antibody at room temperature for 1 h. Detect the signals using Clarity ECL substrate, and quantify using ImageJ (NIH Image, ImageJ 1.52a).

For DNA aging analyse, put the sample in the oven at 70 °C for 15, 30 and 60 min. Then analyze by gel electrophoresis to see the DNA fragmentation. We will estimate the corresponding time at room temperature by extrapolation using the Arrhenius model. And compare with previous reports.

PCR procedure: DNA and DNA pool encoding (kB) of data is amplified with an Agilent Technologies. Add a total volume of 20 μl containing 5 μl sample volume, 10 μl of Kapa Sybr Fast qPCR Master Mix, 3 μL mQ water, 1 μl (10 μM) forward primer and 1 μl (10 μM) reverse primer in to the well. The qPCR for DNA consists of a 3-step amplification protocol (95 °C for 15 s, 56 °C for 15 s and 72 °C for 10 s), and include a duplicates sample. The qPCR for the DNA pool also consists of a 3-step amplification protocol (98 °C for 20 s, 60 °C for 15 s and 72 °C for 20 s), and include a duplicates sample.

DNA concentration measurements: using the plate reader (Epoch; Bio-Tek Instruments, Inc., Winooski, VT, USA) DNA concentration is measured.

Scanning electron micrographs and EDX: spectra is recorded with a SEM/FIB Focused Ion Beam—Nova 200 NanoLab (FEI)) equipped with EDX spectroscopy (Analyzer with an acceleration voltage of 20.0 kV). For this, before and after aging, each dried sample is dispersed on the SEM sample holder and the info is recorded.

Procedure for sequencing: The DNA samples are read by MinION (nanopore) sequencing and decoded to recover the original information. For filtering data and restoring binary information, “Canu” software is used. In the last stage, DNA records are decoded back to digital binary data to confirm the digital data on DNA in dry state. A total read in the MinION expected size is monitored to identify any possible error product during the DNA fragment preparation process.

12,288 bytes of source data is encoded with size 12 KB into oligos and redundancy is check to measure error. To ensure each encoded DNA oligo with the length under the limit of the current synthesis technique (200 nt), the length of encoded DNA sequences set to be around 160 nt after excluding 40 nt for two primer sites. Considering the theoretical mapping potential (the number of bits encoded in one nucleotide) of 1 bits/nt, the length of the binary sequence is 100 bits. To correct errors occurring from any stage in the DNA storage processes, including synthesis, amplification, storing, and sample preparation for sequencing, apply a repeat accumulate (RA) encoding on binary user packets where 5% redundant packets will be generated. With each of the binary packets, 20 bits are used for addressing to order the stochastic oligos and 20 bits are used for redundancy check to detect interior errors in the packet. Each stage involves a correctness check. Afterward, all binary sequences are mapped into DNA sequences to compare the mapping potential (coding potential) and information density of the existing methods. Then the DNA sequences is sent for oligos synthesis. After receiving the synthesized oligos pool, it is amplified using Polymerase Chain Reaction (PCR) before drying/preservation. Then the DNA samples are read by MinION (nanopore) sequencing and decoded to recover the original information. For filtering data and restoring binary information, “Canu” software is used. In the last stage, sequencing data is analysed and decoded to convert the DNA records back to digital binary data to test the efficiency of the digital data on DNA in dry state upon different dry storage formats. A total read in the MinION expected size is monitored to identify any possible error product during the DNA fragment preparation process.

### Example 3 Radiation Protecting DNA Storage Media

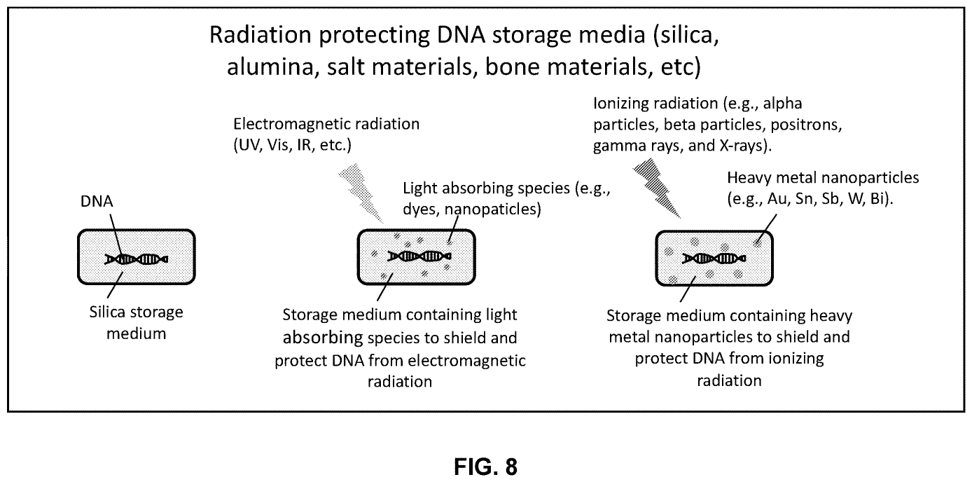

Various storage media (e.g., silica, alumina, salt materials, bone materials, etc.) are evaluated by incorporating light absorbing species such as dyes or nanoparticles (TiO2) in order to shield and protect DNA from electromagnetic radiation (FIG. 8).

### Metal-Organic Frameworks (MOFs)

Metal-organic frameworks (MOFs) feature tunable pore size and high thermal stability [1B]. Moreover, recent advancement of MOFs synthesis offers outstanding biocompatibility and exceptional water stability. For example, ZIF-90 and ZIF-8 MOFs show excellent water stability. [2B] Therefore, MOFs may be suitable for long-term storage of biomolecules like DNA. Furthermore, tunable porosity facilitates incorporation DNA into the pores of MOFs networks and thereafter, MOFs can protect them from organic solvents, pressure, and heating/cooling. In another aspect, the biopolymers in storage may be protected from radiation like X-ray and ultraviolet A (UVA) radiation which can stimulate the reactive oxygen species (ROS) production and damage DNA and other biopolymers. It has been found that cerium oxide nanoparticles (CeO₂) have great potential against UVA and X-ray radiation. [3b] It is reported that cerium oxide nanoparticles (CeO₂) can exhibit an antioxidant effect which can scavenge free radicals. Furthermore, CeO₂ nanoparticles are biocompatible. In another aspect, gold nanoparticles may have high X ray attenuation, and biocompatibility.[4b] Accordingly, in some embodiments, the biopolymers may be stored in a nanoparticle assembly wherein gold nanoparticles are disposed on the CeO₂-coated MOFs to protect the biopolymers from any external radiation and forces.

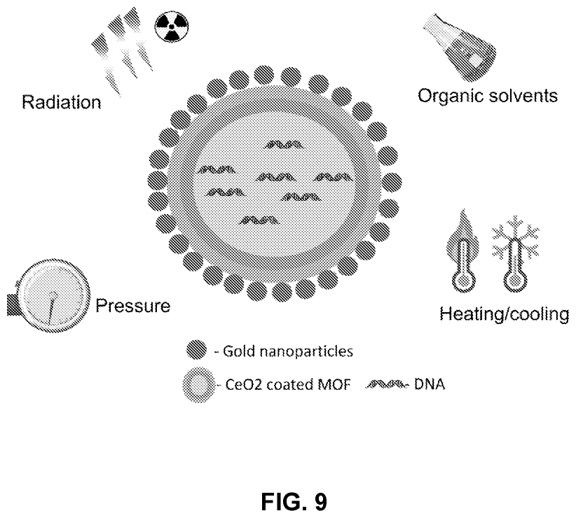

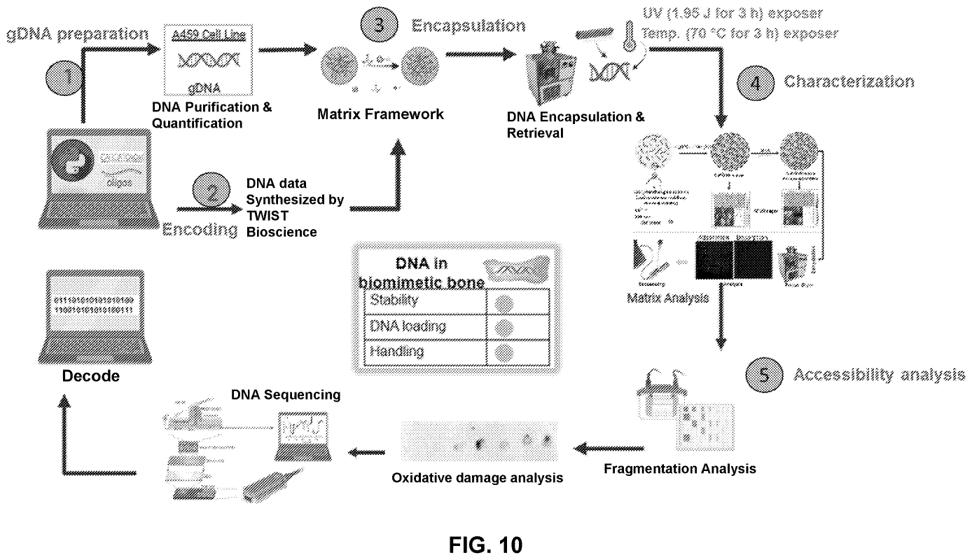

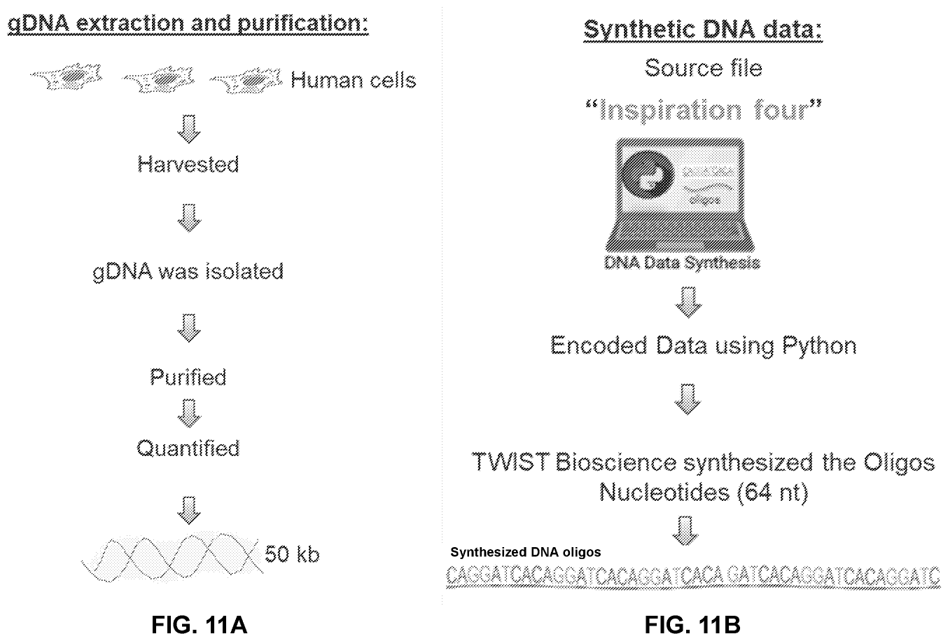

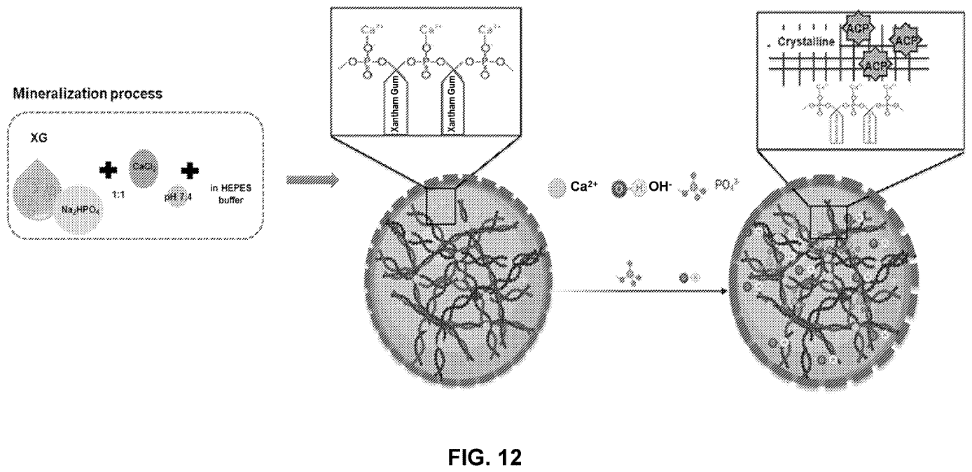

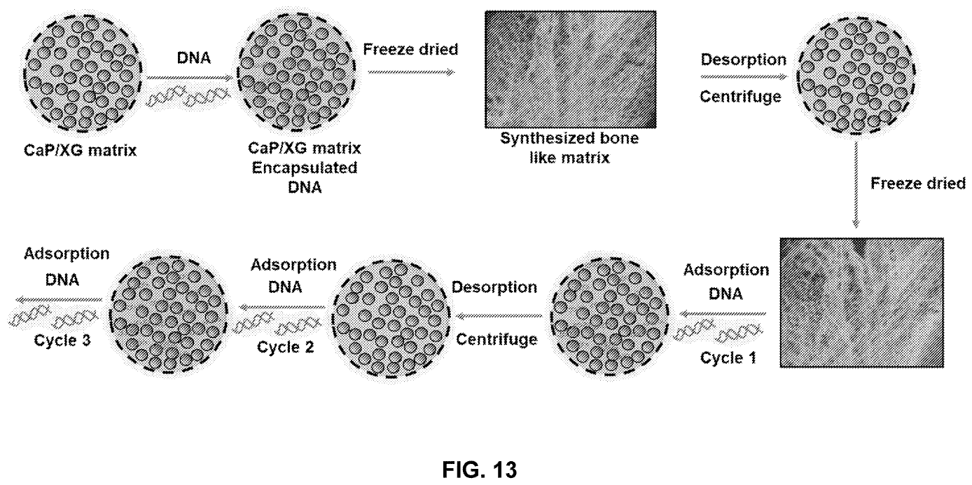

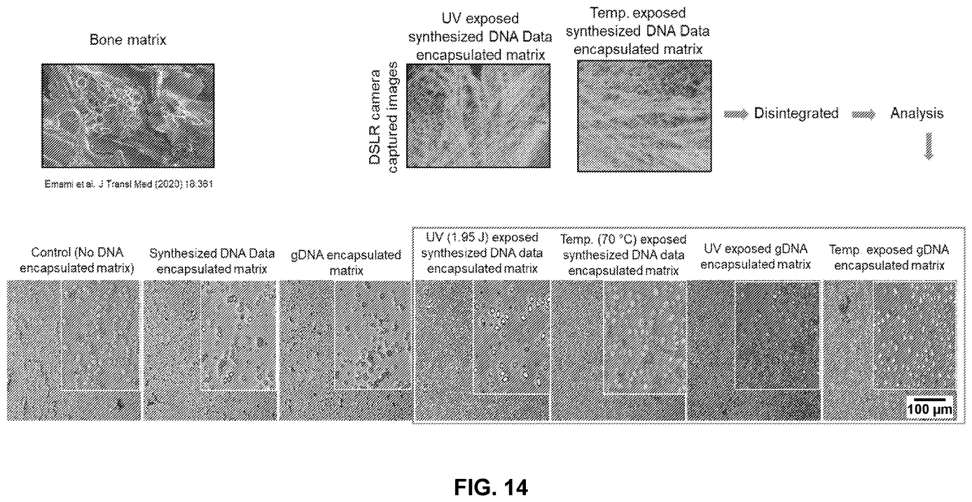

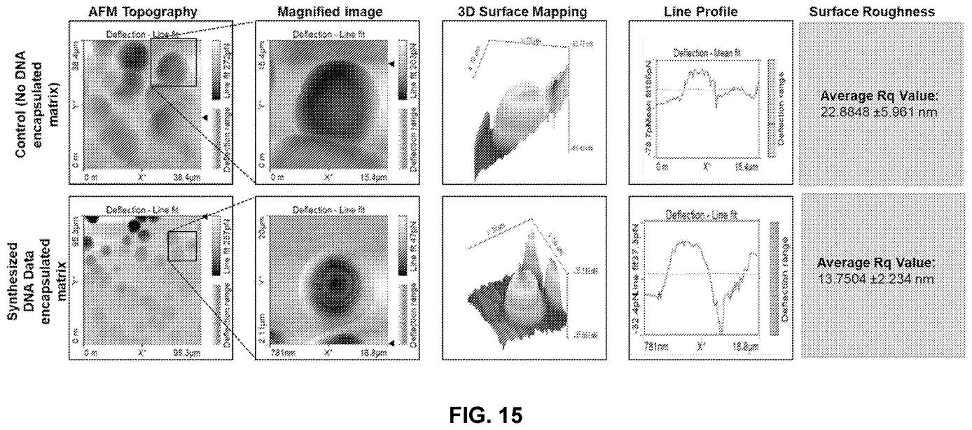

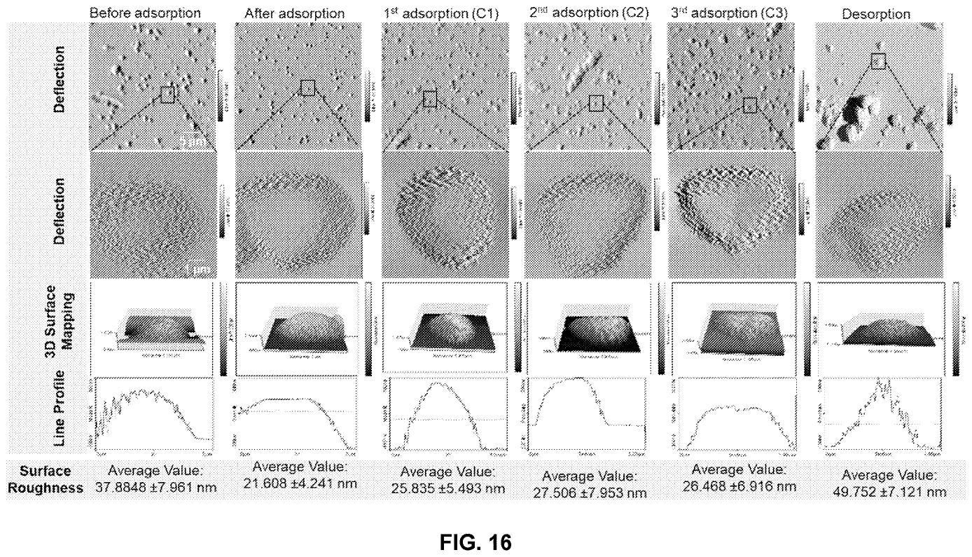

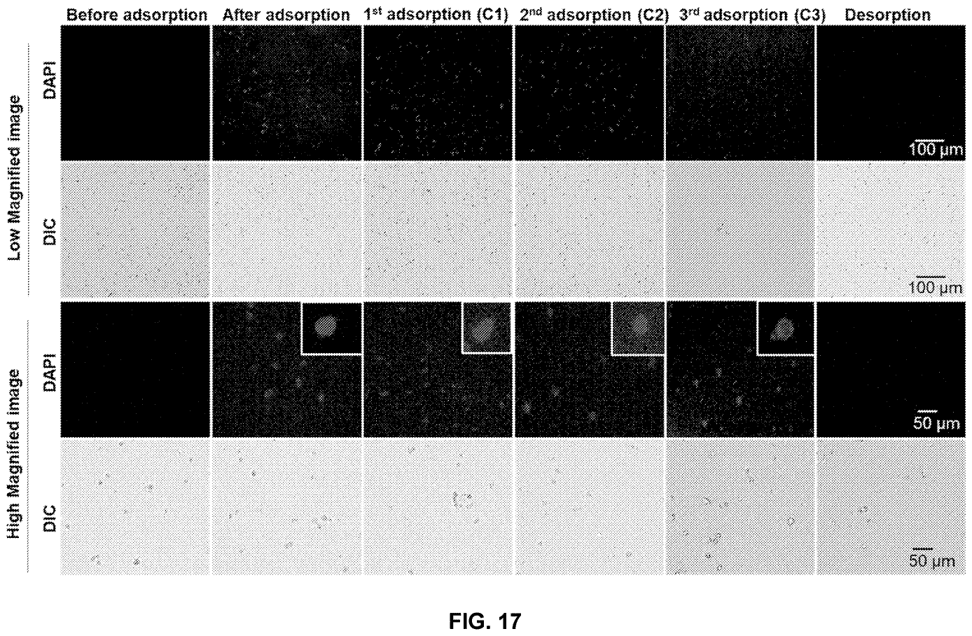

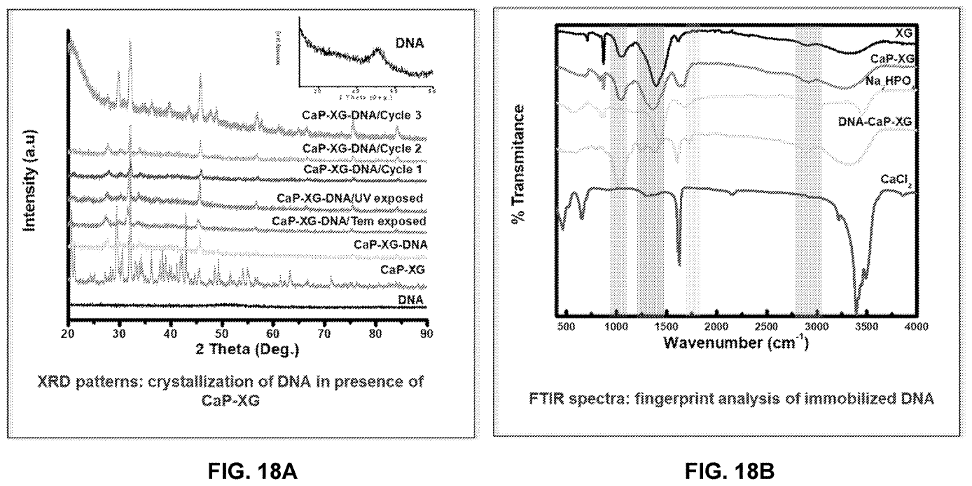

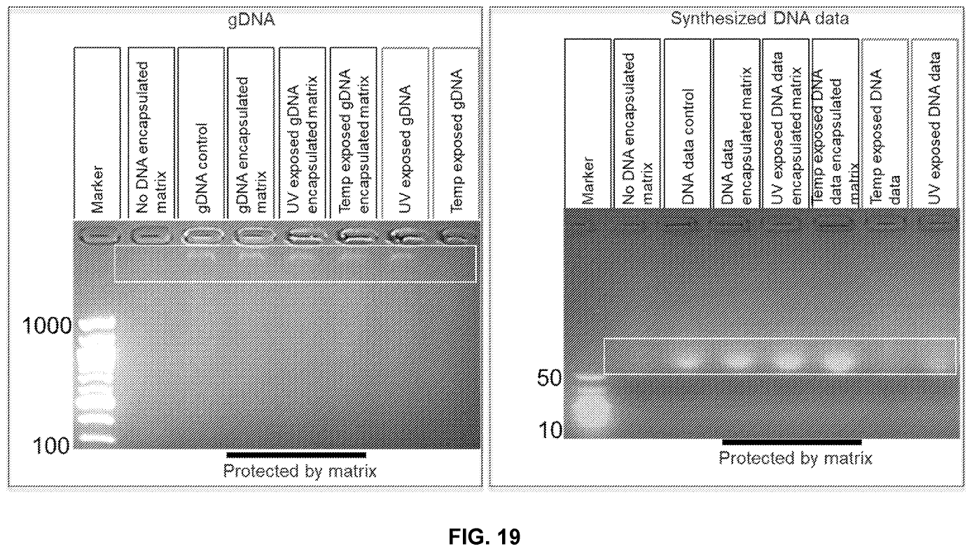

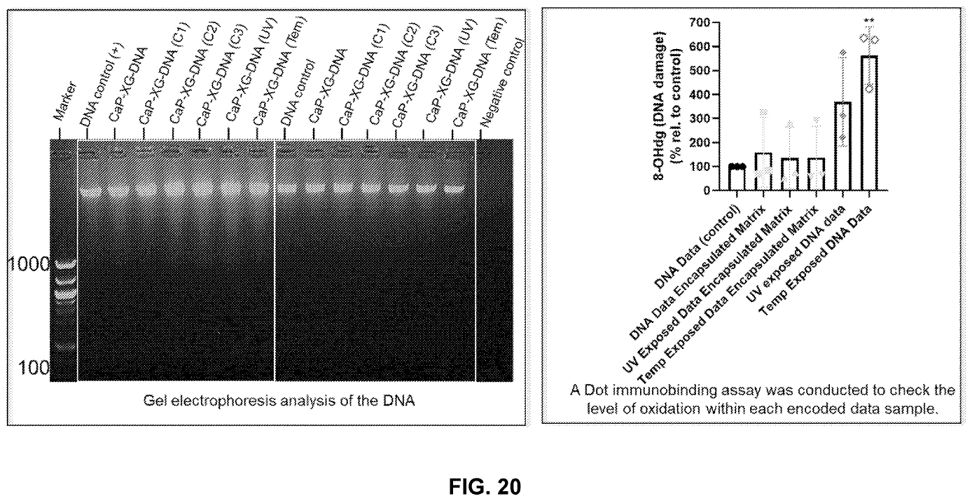

FIG. 9 shows the structure of the nanoparticle assembly which can protect DNA where the MOF (ZIF-90) offers protection of DNA from organic solvents, pressure, and heating/cooling and gold nanostars decorated on the MOF offers long term stability and protection from radiation.

Synthesis of DNA@MOFs—0.5 mg of DNA were added into a solution of 160 mM 2-methylimidazole. 40 mM Zinc acetate dihydrate solution was also prepared. These two solutions were mixed at room temperature for 10 min. Then, the as-synthesized products were centrifuged at 6000 rpm for 10 min, washed with excess D.I. water.

### REFERENCES FOR EXAMPLES 1-3

1. Tan X, Ge L, Zhang T, Lu Z. Preservation of DNA for data storage. Russian Chemical Reviews. 2021/03/01 2021; 90(2):280-291. doi:10.1070/rcr4994
2. Howlett S E, Castillo H S, Gioeni L J, Robertson J M, Donfack J. Evaluation of DNAstable for DNA storage at ambient temperature. Forensic Sci Int Genet. January 2014; 8(1):170-8. doi:10.1016/j.fsigen.2013.09.003
4. Jovanovic N, Bouchard A, Hofland G W, Witkamp G J, Crommelin D J, Jiskoot W. Stabilization of proteins in dry powder formulations using supercritical fluid technology. Pharm Res. November 2004; 21(11):1955-69. doi:10.1023/b:pham.0000048185.09483.e7
5. Allentoft M E, Collins M, Harker D, et al. The half-life of DNA in bone: measuring decay kinetics in 158 dated fossils. Proc Biol Sci. Dec. 7 2012; 279(1748):4724-33. doi:10.1098/rspb.2012.1745
6. Palladino P, Stetsenko D A. New TFA-free cleavage and final deprotection in fmoc solid-phase peptide synthesis: dilute HCl in fluoro alcohol. Org Lett. Dec. 21 2012; 14(24):6346-9. doi:10.1021/ol303124r
7. Jensen E C, Zeng Y, Kim J, Mathies R A. Microvalve Enabled Digital Microfluidic Systems for High Performance Biochemical and Genetic Analysis. JALA Charlottesv Va. Dec. 1 2010; 15(6):455-463. doi:10.1016/j.jala.2010.08.003
8. Yang K, McCloskey C M, Chaput J C. Reading and Writing Digital Information in TNA. ACS Synth Biol. Nov. 20 2020; 9(11):2936-2942. doi:10.1021/acssynbio.0c00361
9. Pinheiro V B, Taylor A I, Cozens C, et al. Synthetic genetic polymers capable of heredity and evolution. Science. Apr. 20 2012; 336(6079):341-4. doi:10.1126/science.1217622
10. Taylor A I, Pinheiro V B, Smola M J, et al. Catalysts from synthetic genetic polymers. Nature. Feb. 19 2015; 518(7539):427-30. doi:10.1038/nature13982
11. Alves Ferreira-Bravo I, Cozens C, Holliger P, DeStefano J J. Selection of 2′-deoxy-2′-fluoroarabinonucleotide (FANA) aptamers that bind HIV-1 reverse transcriptase with picomolar affinity. Nucleic Acids Res. Nov. 16 2015; 43(20):9587-99. doi:10.1093/nar/gkv1057
12. Mei H, Liao J Y, Jimenez R M, et al. Synthesis and Evolution of a Threose Nucleic Acid Aptamer Bearing 7-Deaza-7-Substituted Guanosine Residues. J Am Chem Soc. May 2 2018; 140(17):5706-5713. doi:10.1021/jacs.7b13031
14. Rohland N, Hofreiter M. Ancient DNA extraction from bones and teeth. Nat Protoc. 2007; 2(7):1756-62. doi:10.1038/nprot.2007.247
15. Jiang W, Griffanti G, Tamimi F, McKee M D, Nazhat S N. Multiscale structural evolution of citrate-triggered intrafibrillar and interfibrillar mineralization in dense collagen gels. J Struct Biol. Oct. 1 2020; 212(1):107592. doi:10.1016/j.jsb.2020.107592
16. Thrivikraman G, Athirasala A, Gordon R, et al. Rapid fabrication of vascularized and innervated cell-laden bone models with biomimetic intrafibrillar collagen mineralization. Nat Commun. Aug. 6 2019; 10(1):3520. doi:10.1038/s41467-019-11455-8
17. Kohll A X, Antkowiak P L, Chen W D, et al. Stabilizing synthetic DNA for long-term data storage with earth alkaline salts. Chem Commun (Camb). Mar. 28 2020; 56(25):3613-3616. doi:10.1039/d0cc00222d

### Example 4 Raman Spectroscopy Analysis of Digitally Encoded Biopolymers

Raman spectroscopy offers some distinct advantages over other spectroscopic methods for field analysis. Following laser irradiation of a sample, the observed Raman shifts are equivalent to the energy changes involved in transitions of the scattering species and are therefore characteristic of it. These observed Raman shifts correspond to vibrational transitions of the scattering molecule. Such frequencies, when observed in absorption techniques, occur in the infrared (IR) region of the spectrum are characteristic of the molecules like a ‘spectral fingerprint’. In the Raman technique, the spectrum is in the same spectral region as the exciting laser radiation. As with IR absorption spectroscopy, Raman spectroscopy provides detailed vibrational information, which is often unavailable or unresolved in fluorescence, UV absorption and reflectance spectroscopies. This information can be related to structural changes in complex molecules such as DNA. Raman spectroscopy is also more suitable than IR spectroscopy for biological analysis because it does not suffer from the strong IR absorption band of water. For these reasons, Raman spectroscopy has a great potential for field monitoring where moisture is often present.

### Experiment

### Raman Measurements

Raman measurements of the DNA samples were performed by using a laboratory built portable Raman system which has a 785-nm laser source (Rigaku Xantus TM-1 handheld Raman device), a fiber optic probe (InPhotonics RamanProbe), a spectrometer (Princeton Instruments Acton LS 785), and a charge-coupled device (CCD) camera (Princeton Instruments PIXIS: 100BR_eXcelon). The laser power of the Rigaku Xantus TM-1 system was fixed at 200 mW and the exposure time for the CCD camera exposure was set at 1 second. The Raman measurement was standardized using ethanol.

### Results and Discussion

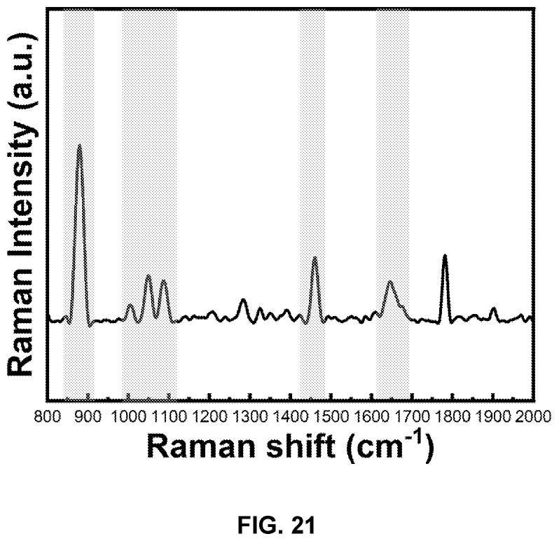

We first performed the Raman measurement of the native DNA sample to obtain a detailed insight into the molecular vibrational fingerprints from which we can correlate the Raman spectra of the DNA samples after thermal and UV-exposer. FIG. 21 displays the background subtracted Raman spectrum of the control DNA sample, which was isolated from the A549 cell line. The Raman spectrum shows that the vibration bands are prominent at 880, 1005, 1057, 1090, 1450, and 1650 cm⁻¹. These vibration bands are mainly the vibration modes of the purine (adenine (A), guanine (G)) and pyrimidine (cytosine (C), thymine (T)) nucleobases. The vibration modes of the DNA nucleobases are summarized in Table 1. The specific Raman signal at 880, 1005, 1057, 1090, 1450, and 1650 cm⁻¹ are for vibration mode of the CO of deoxyribose-sugar moiety, CO (5′) moiety, deoxyribose-sugar moiety, PO₂⁻ phosphate backbone, 5′CH₂ deoxyribose-sugar moiety, and CO of thymine, guanine, and cytosine, respectively. These results are consistent with previously reported results.¹

TABLE 1
Assignment of the vibrational Raman bands of the native DNA.

| Peak position (cm⁻¹) | Assignment |
|---|---|
| 880 | υ(CO)- deoxyribose-sugar |
| 1005 | T, G, C, deoxyribose-sugar |
| 1057 | υ(CO-5′)- deoxyribose-sugar |
| 1090 | υ (PO2⁻) |
| 1450 | δ (5′ CH₂)- deoxyribose-sugar |
| 1650 | υ(C₄═O) - T, G, C |

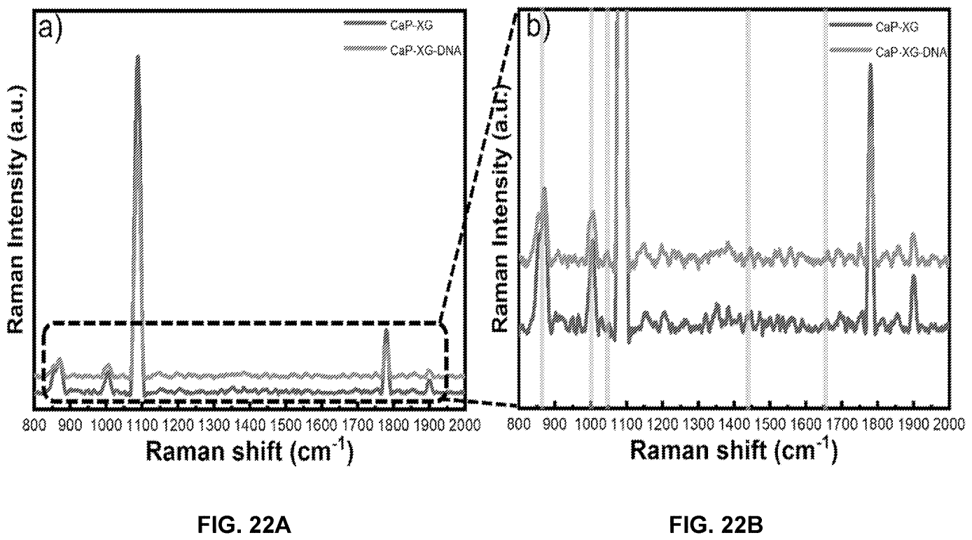

Furthermore, we have performed Raman measurements of the DNA samples after encapsulation with Ca-salts. The Raman spectra were first background subtracted and then analyzed in detail for spectral changes. Interestingly, we can see that the main peaks are at 1090 and 1790 cm⁻¹ indicating a lot of salts are present in the sample as expected (FIG. 22A). Besides these two strong peaks, the Raman spectra also reveal additional minor but noteworthy spectral structures. To extract the valuable vibrational information from the spectrum, we multiplied the intensity scale of original Raman spectrum ten-fold. Since the DNA samples are encapsulated in a calcium phosphate matrix, the spectra exhibit a strong peak at 1090 cm⁻¹, which could be assigned to calcium salt present in the solution. However, even with this intense 1090 cm⁻¹ peak, one can still observe the smaller Raman peaks. FIG. 22B shows the ten-fold zoomed-in Raman spectrum, which reveals the peaks for DNA at 880, 1005, 1057, 1450, and 1650 cm⁻¹. We have further performed the Raman measurements of the samples and we can detect the Raman modes of DNA samples by ten times zoomed in from the original Raman spectra.

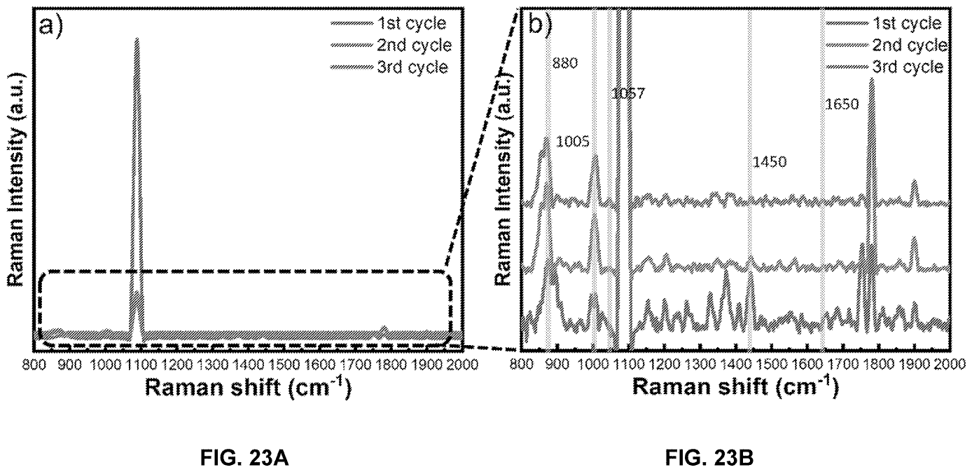

We performed the Raman measurement after three consecutive washing steps. Interestingly, the Raman peak intensity at 1090 cm⁻¹ was reduced gradually from the 1^{st} cycle to the 3^{rd} cycle indicating a reduction of the calcium phosphate matrix (FIG. 23A) from the 1st cycle. However, the calcium phosphate Raman peak is so strong that we could not get the vibrational peak information of the DNA. As mentioned before, we ten-fold zoomed-in on the Raman spectrum (FIG. 23B). When the Raman spectrum is ten-fold zoomed in, we can detect that the peaks at 880 cm⁻¹, 1057 cm⁻¹, 1450 cm⁻¹, and 1650 cm⁻¹, which can be matched with the corresponding control DNA sample. It noteworthy that, we also lose some DNA samples after washing as the Raman peak intensity at 880 cm⁻¹, 1057 cm⁻¹, 1450 cm⁻¹, and 1650 cm⁻¹ are decreased gradually after 1^{st} cycle of washing.

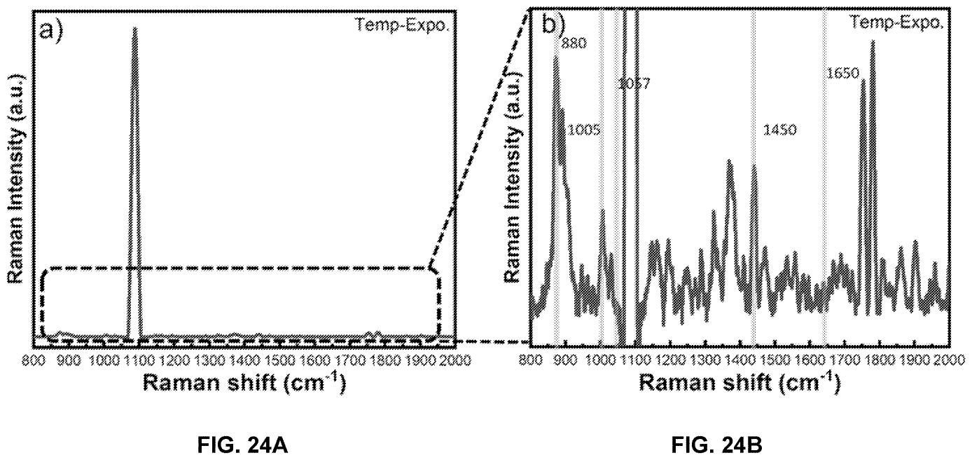

FIG. 24A-B shows the representative Raman and zoomed-in Raman spectra after temperature exposure. Interestingly, we can find the Raman peaks of the DNA at 880, 1005, 1057, 1450, and 1650 cm⁻¹, which indicates that the DNA samples are stable after temperature exposure. It is important to note that DNA is denatured when heated at a high temperature (100 °C or above).^{2, 3} The identical Raman peaks of the temperature exposure and native DNA indicate that the DNA can be stored for the long term even when stored at elevated temperatures.

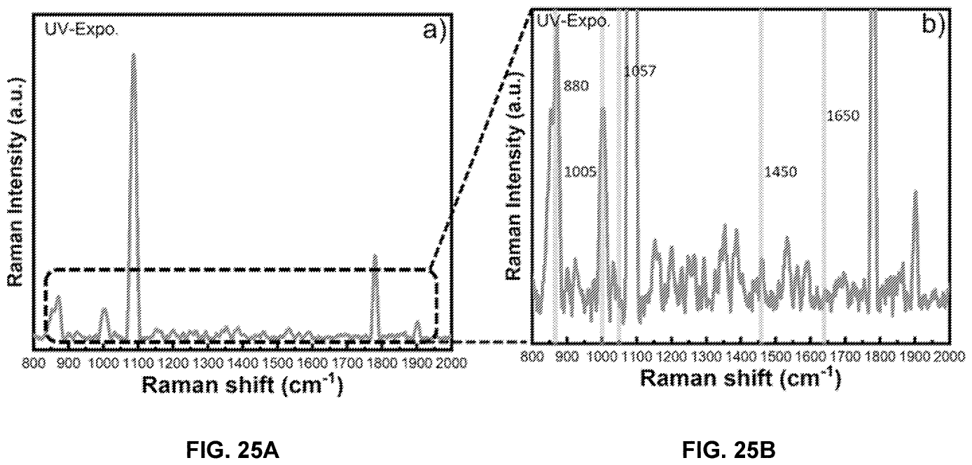

We further investigate the stability of the DNA sample in presence of UV radiation. It is reported that DNA degraded after one hour of UV exposure.⁴ FIG. 25A-B shows the Raman spectra of the DNA sample after UV exposure. The Raman peaks of the DNA sample are identical to the native DNA sample indicating high stability of the DNA sample. This finding indicates that the DNA is stable in presence of UV radiation. Overall, the results indicate that the Raman technique can analyze DNA samples. For future studies, it would be preferable to extract DNA from the matrix before and after treatment to obtain a stronger Raman signal from the DNA without the strong interference from the matrix.

### REFERENCES FOR EXAMPLE 4

1. Chandra, G. K.; Eklouh-Molinier, C.; Fere, M.; Angiboust, J.-F.; Gobinet, C.; Van-Gulick, L.; Jeannesson, P.; Piot, O., Probing in Vitro Ribose Induced DNA-Glycation Using Raman Microspectroscopy. Analytical Chemistry 2015, 87 (5), 2655-2664.
2. Niziot, J.; Ekiert, R.; Kuczkowska, J.; Fryń, P.; Marzec, M., Thermal degradation of biological DNA studied by dielectric spectroscopy. Polymer Testing 2019, 80, 106158.
3. Karni, M.; Zidon, D.; Polak, P.; Zalevsky, Z.; Shefi, O., Thermal Degradation of DNA. DNA and Cell Biology 2013, 32 (6), 298-301.
4. Rahi, G. S.; Adams, J. L.; Yuan, J.; Devone, D. J.-N.; Lodhi, K. M., Whole human blood DNA degradation associated with artificial ultraviolet and solar radiations as a function of exposure time. Forensic Science International 2021, 319, 110674.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Certain molecules disclosed herein may contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every device, system, formulation, combination of components, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A system for storage of digital information via a biopolymer, the system comprising:
a layered microfluidic device comprising:
a pneumatic control layer configured to supply a control gas to a plurality of pneumatically operated valves;
a fluidic layer comprising an interconnected matrix of microfluidic channels, the microfluidic channels being in selective fluid communication with each other via the plurality of pneumatically operated valves, a first side of the fluidic layer being bonded to the pneumatic control layer;
a biopolymer analysis layer comprising solid-state nanopores disposed in a semiconductor support, the solid state nanopores being in selective fluid communication with the interconnected matrix of microfluidic channels, the biopolymer analysis layer being bonded to a second side of the fluidic layer opposite the first side; and
a processor operably connected to the microfluidic device, the processor being configured to:
receive digital information;
design one or more target biopolymer sequences encoding the digital information;
synthesize the one or more target biopolymer sequences via a reaction sequence carried out in the interconnected matrix of microfluidic channels and controlled via the plurality of pneumatically operated valves;
analyze the one or more target biopolymer sequences via the solid-state nanopores;
transfer the one or more target biopolymer sequences to a biopolymer preservation system;
retrieve the one or more target biopolymer sequences from the biopolymer preservation system; and
decode the one or more target biopolymer sequences into the digital information.

2. The system of claim 1, wherein the biopolymer is a nucleic acid and the target biopolymer sequence is a nucleotide sequence.

3. (canceled)

4. The system of claim 1, wherein the supercritical fluid is supercritical nitrogen or supercritical argon.

5. The system of claim 1, wherein the biopolymer preservation system comprises a dehydration channel, the dehydration channel being operably connected to a mineralization medium source and a supercritical fluid source, the processor being configured to cause the one or more target biopolymer sequences to be contacted with the mineralization medium and the supercritical fluid, thereby calcifying and dehydrating the one or more target biopolymer sequences.

6-7. (canceled)

8. The system of claim 1, wherein at least some of the microfluidic channels include one or more magnetic microspheres trapped therein via an externally applied magnetic field, and wherein a surface of each microsphere is functionalized to grow a target biopolymer sequence thereon.

9. The system of claim 1, wherein the digital information comprises binary computer code.

10-12. (canceled)

13. A method for storage of digital information via a biopolymer, the method comprising:
receiving digital information;
designing one or more target biopolymer sequences, the one or more target biopolymer sequences encoding the digital information;
synthesizing the one or more target biopolymer sequences via a reaction sequence carried out in an interconnected matrix of microfluidic channels of a layered microfluidic device, the device comprising;
a pneumatic control layer configured to supply a control gas to a plurality of pneumatically operated valves;
a fluidic layer comprising an interconnected matrix of microfluidic channels, the microfluidic channels being in selective fluid communication with each other via the plurality of pneumatically operated valves, a first side of the fluidic layer being bonded to the pneumatic control layer; and
a biopolymer analysis layer comprising solid-state nanopores disposed in a semiconductor support, the solid state nanopores being in selective fluid communication with the interconnected matrix of microfluidic channels, the biopolymer analysis layer being bonded to a second side of the fluidic layer opposite the first side and controlled via the plurality of pneumatically operated valves;
analyzing the one or more target biopolymer sequences via the solid-state nanopores;
transferring the one or more target biopolymer sequences to a biopolymer preservation system;
storing the one or more target biopolymer sequences in the biopolymer preservation system;
retrieving the one or more target biopolymer sequences from the biopolymer preservation system; and
decoding the one or more target biopolymer sequences into the digital information.

14. The method of claim 13, wherein the biopolymer is a nucleic acid and the target biopolymer sequence is a nucleotide sequence.

15. (canceled)

16. The method of claim 13, wherein storing the one or more target biopolymer sequences comprises contacting the one or more target biopolymer sequences with a mineralization medium.

17. The method of claim 16, wherein the mineralization medium comprises one or more of: Ca₃(PO₄)₂, CaCl₂)·2H₂O, and K₂HPO₄.

18. The method of claim 16, wherein the mineralization medium comprises osteopontin and/or NaOH.

19. The method of claim 16, wherein storing the one or more target biopolymer sequences comprises contacting the one or more target biopolymer sequences in the mineralization medium with a supercritical fluid, thereby forming dehydrated target biopolymer sequences adsorbed on a mineral matrix.

20. (canceled)

21. The method of claim 13, wherein at least some of the microfluidic channels include one or more magnetic microspheres trapped therein via an externally applied magnetic field, and wherein a surface of each microsphere is functionalized to grow a target biopolymer sequence thereon.

22. (canceled)

23. The method of claim 13, wherein the biopolymer is a nucleic acid.

24-26. (canceled)

27. The method of claim 13, wherein the mineralization medium comprises a UV protection agent.

28. The method of claim 27, wherein the UV protection agent is TiO2.

29. The method of claim 13, wherein the mineralization medium comprises an ionizing radiation protection agent.

30. (canceled)

31. The method of claim 13, wherein storing the one or more target biopolymer sequences comprises incorporating the one or more target biopolymer sequences into a nanoparticle system, the nanoparticle system comprising nanoparticles of a metal-organic framework.

32. The method of claim 13, wherein storing the one or more target biopolymer sequences comprises incorporating the one or more target biopolymer sequences into a nanoparticle system, the nanoparticle system comprising nanoparticles of a metal-organic framework coated with CeO₂.

33. The method of claim 31, wherein the nanoparticles comprise a gold layer at least partially encapsulating the nanoparticle.
